(19) United States
(12) Patent Application Publication
(10) Pub. No.: US 2011/0066072 A1
(43) Pub. Date: Mar. 17, 2011

(54) INTRACRANIAL PRESSURE SENSOR

(75) Inventors: Usmah Kawoos, Philadelphia, PA (US); Arye Rosen, Cherry Hill, NJ (US); Harel D. Rosen, Belle Mead, NJ (US); Francis A. Kralick, Philadelphia, PA (US)

(73) Assignee: DREXEL UNIVERSITY, Philadelphia, PA (US)

(21) Appl. No.: 12/558,157

(22) Filed: Sep. 11, 2009

(51) Int. Cl. A61B 5/03; A61M 1/00

(52) U.S. Cl. 600/561; 604/8

(57) ABSTRACT

An intracranial pressure device for measuring CSF pressure in a skull includes a housing located between the scalp and the skull containing pressure device circuitry and a conduit extending downwardly from the housing to the vicinity of the CSF. A pressure sensor is coupled to the conduit and located in communication with the CSF wherein the pressure sensor directly senses the pressure of the CSF and provides a signal representative of the pressure of the CSF to the pressure device circuitry by way of the conduit. The skull has a dura and the conduit extends by way of an opening through the skull and an opening through the dura to position the sensor in direct contact with the CSF. A fluid reservoir can be in communication with the CSF by way of a tube and by way of the housing. The fluid reservoir contains CSF.

### STATEMENT REGARDING FEDERALLY SPONSORED RESEARCH OR DEVELOPMENT

This invention was supported in part by funds from the U.S. government (NIH Grant R21 NS050590-01). The U.S. government may therefore have certain rights in the invention.

### FIELD OF INVENTION

This invention relates to the field of physiological measurements and, in particular, to a system for measuring the presence of a fluid.

### DESCRIPTION OF RELATED ART

The final common pathway for death and permanent disability in head injuries and brain disease is usually increased intracranial pressure. For this reason, measurement and control of intracranial pressure is a major focus of care in these cases, both acutely and chronically. It is known in the art to provide microelectromechanical (MEMS) based microwave intracranial pressure sensing devices which allow for non-invasive monitoring of intracranial pressure when used with a portable microwave monitor. Such devices are useful in several areas. However, existing neurosurgical intracranial monitors can typically be implanted and used only in hospital settings, most typically in operating rooms or intensive care units, and have limited useful life due to drift, infection and other factors.

However, they can be useful in treating many of the head injuries and diseases of the brain, which are major causes of death and disability in the developed nations. Stroke is the third leading cause of death in the United States, and head injury is a leading cause of death in adolescents and young adults. Additionally, according to previous estimates, hydrocephalus can account for 50,000 hospital admissions each year. Between 5,000 and 15,000 people receive a new diagnosis of intracranial tumor, 100,000 have a hemorrhagic stroke, and 1. 5 million have a traumatic brain injury. Clinical determination of intracranial pressure is critical to the management of each of these conditions.

Intracranial pressure can range from approximately −5 to 10 torr in the normal human. Since the skull forms an almost complete rigid container for the brain, measuring intracranial pressure directly can be very difficult. However, penetration of the skull to insert a pressure sensor requires a neurosurgical procedure with significant risks. Thus, measuring intracranial pressure remotely has often been preferable in the prior art. Existing neurosurgical intracranial pressure monitors could only be used in the hospital setting, and have limited useful life due to drift and infection.

A number of neurocranial monitors have been described that are purported to facilitate measurement of intracranial pressure. These devices can be grouped generally into four main categories, namely devices with radiofrequency tuned circuits, devices with vibrating mechanical components, devices with moving magnetic components, and devices with optical components.

However, devices with significant inductive or magnetic components, including radio frequency circuit-based devices, were not compatible with magnetic resonance imaging, a procedure often critical to management of patients with abnormal intracranial pressure. Further, many of these devices had a limited lifetime, particularly devices with plastic components, which age rapidly in vivo when in contact with extracellular space, or slide bearings, which are not reliable over long term. The accuracy of these devices could also be degraded by scar formation and/or requirement for a cerebrospinal fluid (CSF) path. For example, a device relying on measurement via a flexible diaphragm could become useless if encased in relatively stiffer scar tissue while a device requiring CSF flow could become prone to clogging in many cases. In addition, many of these devices required either a large number of parts, precise machining or rare and/or exotic materials making manufacture and assembly cost prohibitive.

Passive (battery-less) Bio-MEMS pressure sensors operating at 10-20 MHz (See DeHennis, A. and Wise, K. D. Digest of IEEE Conference on MicroElectroMechanical Systems, 2002, 252-255), and 330 MHz have been described (See Simons et al., Digest of 2004 IEEE International Microwave Symposium, 2004, 3:1433-1436). These sensors required transcutaneous inductive links for monitoring of the pressure. Since these inductive links operated at near-field, the pressure monitoring receiver was required to be placed on the surface of the body. Accordingly, remote monitoring was not possible. Further, these implants required large inductors, e.g. 3.7 microH (See DeHennis, A. and Wise, K. D. Digest of IEEE Conference on MicroElectroMechanical Systems, 2002, 252-255) and 150-200 nH (See Simons et al., Digest of 2004 IEEE International Microwave Symposium, 2004, 3:1433-1436), which are not compatible with magnetic resonance imaging.

Accordingly, there was a lack of stable, biocompatible, rugged and inexpensive intracranial pressure sensors sufficiently small to be inserted through the burr hole and left inside the cranium following most common neurological procedures, which were compatible with modern imaging techniques including, but not limited to CT, MRI and ultrasound and which monitored intracranial pressure.

Several references disclose the general field of intracranial pressure monitoring. For example, U.S. Pat. No. 4,519,401, issued to Ko, discloses a pressure monitor implant that uses a piezoresistive pressure sensor. The pressure sensor includes a pressure sensing diaphragm, a four-type resistor bridge coupled to the diaphragm and a cavity underneath the bridge for conveying pressure changes. When differential pressure is applied, the diaphragmatic stress produces a differential bridge output proportional to the pressure. A telemeter is used to wirelessly transmit the pressure to a remote receiver. A signal indicative of the pressure is provided to a modulator which is then conveyed to an RF carrier circuit for transmission in the Ko device. An on/off command is also transmitted to the implanted receiver via a 3.5 MHz RF inductive link. The telemeter electronics are contained within a titanium flat pack.

Additionally, U.S. Pat. No. 6,113,553, issued to Chubbuck, discloses an intracranial pressure system that also uses an implantable detector. The Chubbuck device, among other things, is directed at solving the problem caused by changes or drifts of the calibrated baseline (the sensor's zero pressure resonant frequency) during use. This is accomplished using an implantable sensor that includes a resonant circuit comprised of a coil and an adjustable capacitor foamed by a pair of capacitor plates on either side of a bellows filled with a reference gas (e.g., nitrogen) and contained within a chamber. As the cranial pressure changes, the bellows either contracts or expands, thus changing the capacitance and, as a result, changing the resonant frequency of the circuit. Thus, the sensor automatically compares the cranial pressure to the pressure reference contained therein, and outputs a pressure signal indicative of the deviation of cranial pressure from the pressure reference. During operation of the Chubbuck system, a probe is positioned over the scalp where the implantable sensor is located and is subjected to a frequency swept magnetic field (e.g., 25-50 MHz).

U.S. Pat. No. 6,533,733, issued to Ericson, discloses an implantable intracranial pressure monitor which uses an internal power source. A pressure transducer includes a flexible membrane whose deflections are indicative of the cranial pressure. The deflections are measured by extremely low-power strain gauges, or by other conventional strain measurements, such as piezoresistive, optoreflective capacitive. Ericson discloses the use of MEMs technology in such implantable devices to reduce sensitivity to parameters such as attitude, sensor motion, gravity and vibration. It also discloses the transmission of the intracranial pressure monitor data using an on-chip direct sequence spread spectrum wireless RF transmitter operating within an ISM frequency band, such as at 915 MHz. Furthermore, the use of well-known transmission codes, FDMA, TDMA and CDMA, are also discussed. Ericson also teaches the use of even higher frequency bands, e.g., the 2400-2483.5 MHz band, for transmitting intracranial pressure data.

U.S. Pat. No. 6,248,080 (Miesel, et al.) also discloses an implantable medical device which includes a sensor that is preferably implanted in the brain itself, not the cranium. The sensor includes a battery for powering internal circuitry. The sensor circuitry includes a pickoff capacitor Cₚ whose plate spacing varies with cranial pressure. A pressure and temperature signal modulating circuit translates the pressure and temperature modulated pickoff and reference capacitor Cₚ and C_{R} values into charge time-modulated intervals T_{PRS} and Tₜₑₘₚ. These are used in conjunction with barometric pressure values to generate and wirelessly transmit intracranial pressure monitor values.

U.S. Patent Publication No. 2002/0177782, filed by Penner, discloses systems and methods for measuring pressure in a sealed or isolated system by converting or correcting data received from the system using one or more remote databases. A variety of implants are shown that may use a biosensor or an actuator or both. The implants can be controlled from, and can communicate with, a remotely-located controller. MEMs-based sensors are also disclosed in Penner.

U.S. Pat. No. 5,797,403, issued to DiLorenzo, teaches a method for the reduction of fluid in order to control edema in a system for use during neurosurgical procedures. Additionally, a hyperbaric chamber is affixed to the head to apply pressure to an exposed cerebral surface. A positive pressure in the pressurized chamber is selected to reduce or reverse an intracranial pressure gradient born by an exposed cortical surface. The positive pressure in the chamber is maintained by a positive fluid pressure applied to an inflow port of the chamber. An outflow port permits circulation of fluid within the operative zone.

U.S. Patent Publication No. 2001/0027335, filed by Meyerson, discloses a device for monitoring a patient recovering from intracranial surgery. Meyerson discusses methods for measuring intracranial pressure. The methods discussed by Meyerson breach the skull and have varying degrees of invasiveness. They include ventriculostomy, intraparenchymal fiberoptic catheters, epidural transducers, subdural catheters, and subdural bolts. Additionally, Meyerson addresses a drawback common to all of the foregoing methods, the need to calibrate the devices used in the techniques. Furthermore, Meyerson addresses the fact that all of the foregoing techniques must be performed by highly trained specialists, usually neurosurgeons, within clinical settings usually available only in the larger medical facilities. Thus, a portable system purported to be suitable for monitoring intracranial pressure under circumstances encountered by first responders is disclosed.

The interfaces to the patient required by the Meyerson system include a plug transducer or a tube inserted into the ear, a pulse oximeter clip on the finger or ear lobe, and a band around the thorax to detect respiration. These devices permit collection of data useful for determining intracranial pressure that can be downloaded when the patient arrives at a hospital. This technique can also be used to collect data in a home environment and transmit it to a hospital. Data useful for this purpose can also be accumulated from, for example, a lumbar puncture if a more invasive technique is appropriate. While the Meyerson system allows measurement of parameters related to intracranial pressure outside of a hospital environment, it does not directly measure the intracranial pressure or provide any means for controlling the pressure prior to the time the patient reaches the hospital.

U.S. Pat. No. 5,836,935, issued to Ashton, discloses an implantable, refillable, rate controllable drug delivery device with a hollow reservoir, and a drug delivery tube communicating with the hollow reservoir. Once implanted, a tubular portion of the device provides continuous access to an internal region of the body without requiring additional needle penetrations into the regions. Thus, the tubular portion can serve as a continuously available conduit for fluids, such as fluids containing drugs, which can be injected into or withdrawn from the tubular portion by a syringe. A rate-limiting membrane can release the injected drugs at a controlled rate.

U.S. Patent Publication No. 2004/0262645, filed by Huff, discloses a phased-array antenna system using RF (radio frequency) devices composes of MEM switches and low-temperature co-fired ceramic technology. It also discloses the use of a pressure sensor in an intracranial pressure measurement application. U.S. Pat. No. 7,025,739, issued to Saul, teaches an intracranial pressure monitor within a system for draining cerebral spinal fluid in order to control intracranial pressure. U.S. Pat. No. 4,354,506, issued to Sakaguchi, teaches a simple intracranial pressure gauge including a powerless resonance circuit and a pressure sensitive section which can be implanted under the scalp. U.S. Pat. No. 5,291,899, issued to Wantanabe, teaches a method for measuring intracranial pressure without calibration wherein a valve capable of communicating with the atmosphere is closed following a zero point correction of a pressure transducer. U.S. Patent Publication No. 2005/0137578, filed by Heruth, teaches an implantable catheter having infusion sections with permeable membranes and control valves for delivering drugs to internal organs.

An implantable intracranial pressure sensing device was described in WO 2007/065140, PCT/US2006/061451, filed by Samuel R. Neff. Neff described a MEMS-based microwave device sized for implantation into a cranium through a burr hole during a neurosurgical procedure. The device included a chip with an oscillator and an oscillator bias control circuit, a microwave antenna coupled to the oscillator output, a sensing component, preferably an MEMS capacitor and a power source. The capacitance of the MEMS capacitor varies with the intracranial pressure changes, thereby changing the oscillation frequency of the oscillator. Furthermore, the Neff device included a portable microwave monitor for display and external monitoring of the oscillator output transmitted via the antenna of the device and received by the portable microwave monitor.

The Neff system was useful for implantation inside a cranium during a neurosurgical procedure, or during other circumstances occurring within a hospital setting, for example in an intensive care unit. It also included a unit operating at microwave frequencies so that it had the necessary frequency sensitivity to the change in its tank capacitor, and the ability to detect the microwave signal transmitted by a small antenna inside the implant from a significant distance outside the patient. An ISM band microwave frequency of 2.4 GHz was used. Sensing components, electronics and an antenna were assembled on printed circuit boards constructed of silicon dioxide or aluminum oxide substrate. For biocompatibility, it was coated with a very thin layer of parylene (polymerized para-xylylene).

The sensing component could be an oscillator operating at the Industrial-Scientific-Medical (ISM) band of 2.4000-2.4835 GHz for a pressure range of −25 to 200 ton, which corresponded to about S=0.37 MHz/torr sensitivity. It could also include a CMOS chip, fabricated by a submicron CMOS process, including the oscillator and an oscillator bias control circuit since CMOS is a commercially available, low-power consuming technology. Further, the CMOS oscillator was based on a differential cross-coupled topology (Razavi, B. in Design of Integrated Circuits for Optical Communications, New York, McGraw Hill 2002). The CMOS chip also included a bias control circuit, for example a CMOS timer, to save battery power.

An alternative to the MEMS capacitor sensing component was a piezoresistive pressure sensor. In this approach, a piezoresistive sensor output was applied through signal conditioning circuitry to a tuning voltage of a voltage controlled oscillator. The oscillator output was coupled to an antenna which transmitted the output to an external monitoring and/or display unit. An example of an antenna used in such systems is the 2.4 GHz Bluetooth chip antenna 2.2 mm×6.5 mm2 in size (LINX Technology). This type of antenna was fabricated on a very high dielectric constant substrate, and mounted on a printed circuit board as a surface mount component. The antenna had an input impedance of 50 ohms and 3 dB bandwidth of 180 MHz.

Power to the device was preferably provided via small rechargeable batteries such as 3 volt, 30 mAh capacity, lithium battery. The DC current and consumed power could be 11.5 mA and 34 mW. Batteries such as these could be recharged by way of an inductive link which required placement of a planar coil in the vicinity the antenna. It was sized sufficiently small so that is could be implanted through a burr hole. Additionally, a photodiode array could be provided for recharging the battery. The Neff device could be packaged within a titanium cylinder for ruggedness and biocompatibility. A Teflon window and a biomedical grade silicone sealant could be provided.

Intracranial pressure measurement is also widely discussed in the non patent literature. For example “Continuous intracranial pressure monitoring via the shunt reservoir to assess suspected shunt malfunction in adults with hydrocephalus,” by Geocadin, Neurosurg. Focus/Volume 22/April, 2007, discloses in-hospital studies of continuous monitoring of a sample of patients with CSF shunts. In the Geocadin study shunt reservoirs were provided under the scalps of the patients. The reservoirs were monitored to detect overdrainage, underdrainage or variable drainage. Drainage was adjusted according to the monitoring of the reservoir, and the effects of the adjustments on the outcome of the patients' treatment were assessed.

The use of a reservoir to evaluate shunt malfunction is also disclosed in “Evaluation of shunt malfunction using shunt site reservoir,” by Sood, Children's Hospital of Michigan, 1016-2291/00/0324-0180, 2000. In the system disclosed by Sood, a ventricular catheter was placed alongside a proximal catheter of the shunt and connected to a subgaleal reservoir. When a shunt malfunction was suspected, a standard shunt function evaluation was performed using a shunt tap, a CT scan or a shunt injection, and the pressure from a tap of the reservoir was obtained.

However, the systems taught by Geocadin and Sood, like the preceding references, were not suitable for emergency implantation in a patient under adverse conditions such as the those existing at the scene of an accident or on a battlefield, in order to monitor and control intracranial pressure until more sophisticated facilities were available.

All references cited herein are incorporated herein by reference in their entireties.

### SUMMARY OF THE INVENTION

An intracranial pressure device for measuring CSF pressure in a skull having a scalp includes a housing located between the scalp and the skull containing pressure device circuitry and a conduit extending downwardly from the housing to the vicinity of the CSF. A pressure sensor coupled to the conduit and located in communication with the CSF is also included wherein the pressure sensor senses the pressure of the CSF and provides a signal representative of the pressure of the CSF to the pressure device circuitry located in the housing by way of the conduit. The skull has a dura and the conduit extends by way of an opening through the skull and an opening through the dura to position the sensor in direct contact with the CSF.

A fluid reservoir can be in communication with the CSF by way of a tube. A fluid reservoir can be in communication with the CSF by way of the housing. The fluid reservoir contains CSF. The fluid reservoir is self sealing after it is penetrated by a syringe to prevent fluid from passing through a wall of the fluid reservoir at a point where the fluid reservoir is penetrated by the syringe. Fluid is withdrawn from the fluid reservoir by way of a syringe. Fluid is injected into the fluid reservoir by way of a syringe. The fluid is withdrawn from the fluid reservoir in accordance with the signal representative of the pressure of the CSF. The sensor is MEMS-based circuitry. The pressure device circuitry comprises transmission circuitry and a transmission signal is transmitted by the transmission circuitry in accordance with the signal representative of the pressure of the CSF. A display is provided in accordance with the transmission signal. The pressure device circuitry includes a battery and an energy source for recharging the battery. The energy source can be an energy transducer. The energy transducer can be a photodetector for converting light energy into electrical energy. A substrate can carry a plurality of energy transducers. The energy transducer can be attached to the housing portion. The energy transducer is located interior to at least a layer of the skin.

The intracranial pressure monitoring device of the invention can be formed in a shape suitable for locating the sensor subdurally to make contact with the cerebrospinal fluid and can include a reservoir. Furthermore, the reservoir can be an elastic bladder located below the scalp and outside the skull which is fed by a tube extending down into the cerebrospinal fluid. A one-way mechanical valve can be provided at some point along the length of the tube. The reservoir can be palpated from the exterior of scalp to determine when fluid has accumulated therein. The fluid can be removed by piercing the scalp and the reservoir with a syringe and drawing the fluid out. The material of the reservoir is selected to be elastically self-sealable. This feature better adapts the monitor to adverse conditions such as accident scenes or battlefield conditions in which surgical implantation, such as surgical implantation of a shunt to deposit the fluid in other body cavities, is not feasible.

The sensor can be attached to a probe-like extension that can extend from the vicinity of the scalp or the skull, through an opening in the skull, through the dura, and down to the level of the CSF. The probe can include a pressure sensor attached thereto and positioned to communicate directly with the CSF. The subdural sensor of the invention can thus directly sense the pressure of the CSF. Accordingly, the subdural sensor can provide a direct reading of the pressure of the CSF. A housing, such as a disc shaped housing, can contain the other elements of the intracranial pressure monitoring system, such as the battery and the electronics, and any other required components. The housing can be placed outside the skull, immediately below the scalp. In an alternate embodiment the housing can be place outside the scalp. The opening extending from the housing to the CSF can be very narrow, since it only needs to accommodate the sensor and the probe. For example, a diameter of 2-4 mm can be more than sufficient. The fact that only a very narrow opening through the skull and dura is required, and that only the probe needs to be extended through the opening, facilitates implanting the intracranial pressure monitoring system under adverse conditions.

Additionally, a battery and a system for keeping the battery charged can be provided. A flexible substrate can be implanted beneath the surface of the scalp. The implanted substrate can be provided with one or more energy transducers for converting light energy into electrical energy. The electrical energy from the energy transducers can trickle charge the batteries in the monitor. The energy transducers can be located on the outside of the scalp or implanted beneath the surface of the scalp. If they are implanted beneath the surface of the scalp they should not be implanted so deep that light cannot pass through the tissue to the energy transducers to be converted to electrical energy in order to charge the battery. Alternately, the electrical energy can be used to charge a capacitor that will run the device for a short duration of time, which is just enough to record the measurements from the device. Thus, the capacitor can eliminate the need for a battery. The energy transducers can be photodetectors, photodiodes, photocells, solar cells, etc. Additionally, in one embodiment the energy transducers can be coated with parylene. A coating layer of parylene having a thickness of about 2.5 microns does not substantially alter the efficiency of the energy conversion of the energy transducers.

The present invention can include a MEMS-based microwave intracranial pressure device sized for implantation into the cranium through a burr hole under adverse conditions. The device can include a chip with an oscillator and an oscillator bias control circuit, a microwave antenna coupled to the oscillator output, a sensing component, preferably an MEMS capacitor, whose variation with the intracranial pressure changes the oscillation frequency of the oscillator, and a power source. In a preferred embodiment of the invention the intracranial pressure device is mass producible.

Additionally, a preferred embodiment of the invention is an intracranial pressure measuring device including a reliable and mass-producible MEMS-based microwave intracranial pressure sensing component. A portable microwave monitor for display and external monitoring of the output transmitted via an antenna coupled to the device can be included. The transmitted output can be received by the portable microwave monitor and displayed.

Thus, a reliable and mass-producible MEMS-based microwave intracranial pressure sensing device for use with a portable microwave monitor and methods for non-invasively monitoring and controlling intracranial pressure with this device under adverse conditions are provided.

The system and method of the invention can be particularly advantageous when used in the field of neonatal hydrocephalus. Neonatal hydrocephalus can be described as either communicating hydrocephalus, also called non-obstructive hydrocephalus, or as non-communicating hydrocephalus, commonly called obstructive hydrocephalus.

In non-obstructive hydrocephalus, the drainage catheter may be placed either in the ventricles themselves (ventricular catheter) or positioned in the subarachnoid space. In obstructive hydrocephalus, however, intraventricular pressure is increased, but no communication of fluid flow occurs to the remainder of the cerebrospinal fluid system. As a result, the end of the drainage catheter can be placed within the ventricular system.

Elevations in neonatal intracranial pressure, can be relieved by one of two biomedical techniques. The placement of a ventricular reservoir offers a temporary means of withdrawing cerebrospinal fluid. Tubing is inserted into the ventricle, and is attached proximally to a subcutaneous reservoir. This reservoir is intermittently decompressed by transdermal insertion of a hypodermic needle, and fluid removal via attached syringe. However, if long-term drainage is needed, a ventriculo-peritoneal (VP) shunt (with or without an integral reservoir) is surgically installed. As with a simple reservoir, one end of the drainage tubing is inserted into the ventricle. Tubing is then tunneled subcutaneously to terminate in the peritoneal cavity, allowing continuous drainage of cerebrospinal fluid.

### BRIEF DESCRIPTION OF THE DRAWINGS

FIG. 1 shows an embodiment of the intracranial pressure monitor of the invention.

FIG. 2 shows an alternate embodiment of the intracranial monitor of the invention.

FIG. 3 shows an alternate embodiment of the intracranial pressure monitor of the invention.

FIG. 4 shows a system suitable for use in powering the intracranial pressure monitor of the invention.

FIG. 5 shows an alternate embodiment of the pressure monitor of the invention.

FIG. 6 the receiving and feedback subsystems of the alternate embodiment of FIG. 4.

### DETAILED DESCRIPTION OF THE INVENTION

Referring now to FIG. 1, there is shown an embodiment of the intracranial pressure monitor system 10 of the invention. The intracranial pressure monitor system 10 includes a housing portion 18, which can contain a portion of the circuitry required for monitoring the pressure of the CSF within a skull 14. The circuitry within the housing portion 18 of the intracranial pressure system 10 can be substantially similar to the circuitry disclosed in U.S. Pat. Pub. No. 2009/0216149, entitled “Self-Contained, Implantable, Intracranial Pressure Sensing Device and Methods For Its Use In Monitoring Intracranial Pressure,” published Aug. 27, 2009, which is incorporated by reference in its entirety. In a preferred embodiment of the invention, the housing portion 18 can be located between the scalp 16 and the skull 14, with the bottom surface of the housing portion 18 resting on the outer surface of the skull 14. In another embodiment, the bottom surface of the housing portion 18 can rest on the outer surface of the scalp 16.

The circuitry and methods for monitoring intracranial pressure can be substantially similar to those described in “In-Vitro and In-Vivo Trans-Scalp Evaluation of an Intracranial Pressure Implant at 2.4 GHz,” by Usmah Kawoos, Mohammad-Reza Tofighi, Ruchi Warty, Francis A. Kralick and Arye Rosen, IEEE Transactions on Microwave Theory and Techniques, Vol. 56, No. 10, pp. 2356-2365, October 2008, which is incorporated by reference in it entirety. Additional disclosure is provided in “Characterization of Implantable Antennas for Intracranial Pressure Monitoring Reflection by and Transmission Through a Scalp Phantom,” by Ruchi Warty, Mohammad-Reza Tofighi, Usmah Kawoos and Arye Rosen, IEEE Transactions on Microwave Theory and Techniques, Vol. 56, No. 10, pp. 2366-2375, October 2008, which is also incorporated by reference in it entirety. Differences between the intracranial pressure monitoring systems in the incorporated references and the instant invention are discussed hereinbelow.

A probe like extending portion 32 extends downwardly from the bottom of the housing portion 18. The housing portion 18 and the extending portion 32 are preferably foamed of a relatively inert metal, such as titanium, aluminum or stainless steel. The extending portion 32 extends through an opening which is drilled through the skull 14 and through the layers between the skull 14 and the brain, including the dura 38. The extending portion 32 can thus extend into the subarachnoid space 44, which contains the CSF being monitored by the intracranial pressure monitor system 10. The opening through the skull 14 and the dura 38 can be very small, as described in more detail below.

The relatively small size of the required opening, and the fact that the scalp 16 can be stitched closed over the housing portion 18 (as shown by scalp stitches 24) after inserting the intracranial pressure monitoring system 10, permit the insertion of the intracranial monitoring system 10 under relatively adverse conditions, rather than requiring a hospital environment. Thus, in emergencies, the intracranial monitoring system 10 can be inserted at accident scenes or even on battlefields. Additionally, the intracranial pressure monitor system 10 can therefore be inserted by trained first responders, rather than requiring the patient to wait until a more highly skilled surgeon is available.

A sensor 34, for example a MEMS capacitor which can serve as a pressure sensor, is provided in the vicinity of the bottom of the extending portion 32. The sensor 34 is preferably on the bottom surface of the extending portion 32. Accordingly, the sensor 34 is located below the dura 38 and within the subarachnoid space 44. In a preferred embodiment, a pressure sensor 34 is thus placed within the subarachnoid space 44 in direct contact with the CSF. The sensor 34 can be placed on an upper surface of the CSF, or the tip of the extending portion 32, and hence the sensor 34, can be immersed in the CSF. The directly sensed pressure of the CSF within the intracranial pressure monitor system 10 is therefore a very accurate measurement of the actual pressure of the CSF. The extending portion 32 can serve as a conduit for transmitting an electrical signal representative of the pressure of the CSF from the sensor 34 to the housing portion 18, for example by way of a wire (or a coaxial cable) or a plurality of wires running the length of the extending portion 32.

All of the circuitry of the intracranial pressure monitor system 10, other than the sensor 34, can reside within the housing portion 18. The circuitry in the housing portion 18 can include, for example, a battery or a capacitor that can be charged for providing power to the circuitry. Additionally, a high frequency transmitter and an antenna for transmitting a signal representative of the pressure of the CSF to the exterior of the scalp 16 and any other circuitry can be included. The pressure signal transmitted to the exterior of the scalp 16 can be received and displayed. Since the extending portion 32 must only carry the very small sensor 34, and means for transmitting a signal from the sensor 34 to the housing portion 18, it is possible to use an extending portion 32 having a very small diameter. For example, the diameter of the extending portion 32 can be 2-4 mm or less.

The length of the extending portion 32 depends on the thickness of the skull 14 of the person or animal receiving the intracranial pressure monitor system 10. For a typical adult, a downwardly extending length of about 3-6 mm can be sufficient for the tip of the extending portion 32 to reach from the outside of the skull 14 to the subarachnoid space 44, and make direct contact with the CSF, to provide a direct measurement of the CSF pressure. The housing portion 18 is preferably disc shaped or cylindrical such shapes can fit easily between the scalp 16 and the skull 14. However, the housing portion 18 can be any shape that may be desired or convenient. The diameter and thickness of the housing portion 18 can depend on the amount of circuitry required. For a typical adult human a housing portion 18 diameter of about 2 cm can be sufficient to house the required circuitry (which includes the battery). In the absence of a battery, the diameter of the housing can be about 1 cm.

Additionally, in a preferred embodiment of the intracranial pressure monitoring system 10, a reservoir 22 can be provided. The reservoir 22 can be located between the scalp 16 and skull 14, preferably in the vicinity of the housing portion 18. The reservoir 22 is an elastic bladder that can include a tube 40. The tube 40 can be in fluid communication with the interior of the reservoir 22 at one end, and with the CSF within the skull 14 at the other end. In order to provide fluid communication between the interior of the reservoir 22 and the CSF, the tube 40 can pass through the same opening in the skull 14 and the dura 38 as the extending portion 32 which extends downwardly from the housing portion 18. The fluid communication between the interior of the reservoir 22 and the CSF permits the CSF to flow from the subarachnoid space 44 through the tube 40 to the reservoir 22. In a preferred embodiment, the tube 40 can have a valve for selectably blocking and unblocking the flow of CSF between the subarachnoid space 44 and the reservoir 22.

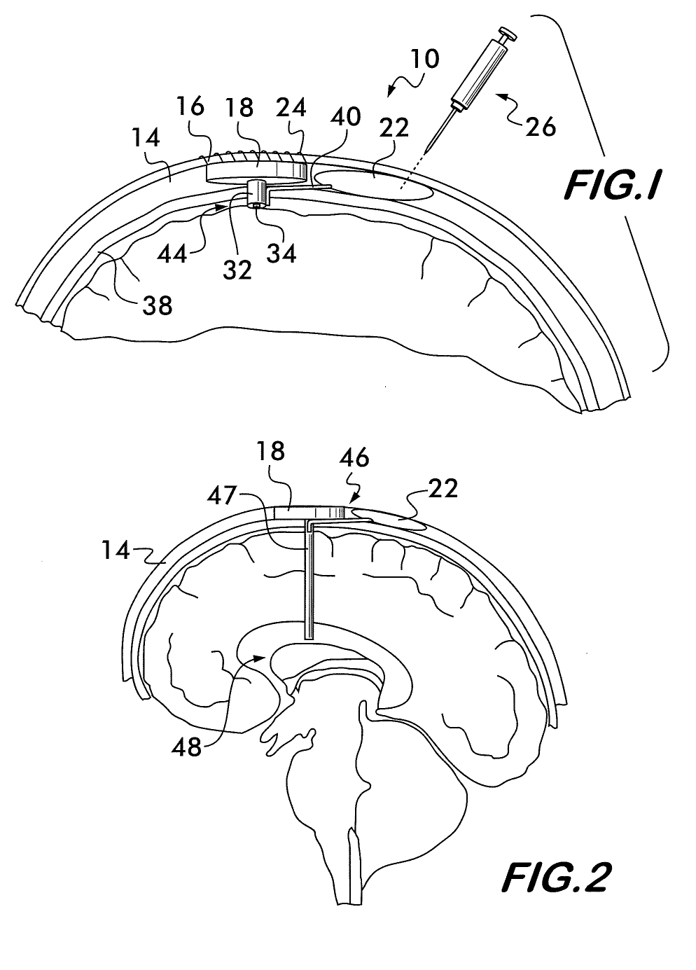

Referring now to FIG. 2, there is shown the intracranial pressure monitor system 46. The intracranial pressure monitor system 46 includes the housing portion 18 having an extending portion with a sensor attached to its tip as previously described. The housing portion 18 can include all of the circuitry of the system 46, with the exception if the sensor at the tip of the extending portion and the electrical connection between the sensor and the circuitry within the housing portion 18. The intracranial pressure monitor system 46 also includes the reservoir 22. The reservoir 22 is in fluid communication with the CSF by way of the tube 47. However, in the embodiment of the intracranial pressure monitor system 46, the tube 47 can extend to the CSF within the ventricular system. This is useful when obstructions prevent the CSF from flowing from the intraventricular region to the remainder of the CSF system, causing the intraventricular pressure to increase. Fluid from the intraventricular region can be shunted to a subcutaneous region in one embodiment.

The fluid within the reservoir 22 can be palpated from outside the scalp 16. This can permit a user to make an approximate estimate of state of the CSF, for example, the pressure of the CSF. Additionally, the material forming the reservoir 22 can be a self sealing elastic to permit it to seal itself after being penetrated by a syringe 26. The self sealing property of the material forming the reservoir 22 can prevent fluid from passing through an opening in a wall of the reservoir 22 created by the syringe 26 when the syringe 26 penetrates the reservoir 22. For example, the material forming the reservoir 22 can be a silastic substance containing polymeric silicones. These substances are suitable because they have many of the properties of rubber, but are more capable of withstanding a wide range of temperatures and other causes of deterioration. Accordingly, the syringe 26 can be used to withdraw CSF from the reservoir 22 in order to avoid or alleviate CSF overpressure within the skull 14. Additionally, the syringe 26 can be used to inject fluid directly into the CSF, for example fluid containing drugs, by way of the reservoir 22.

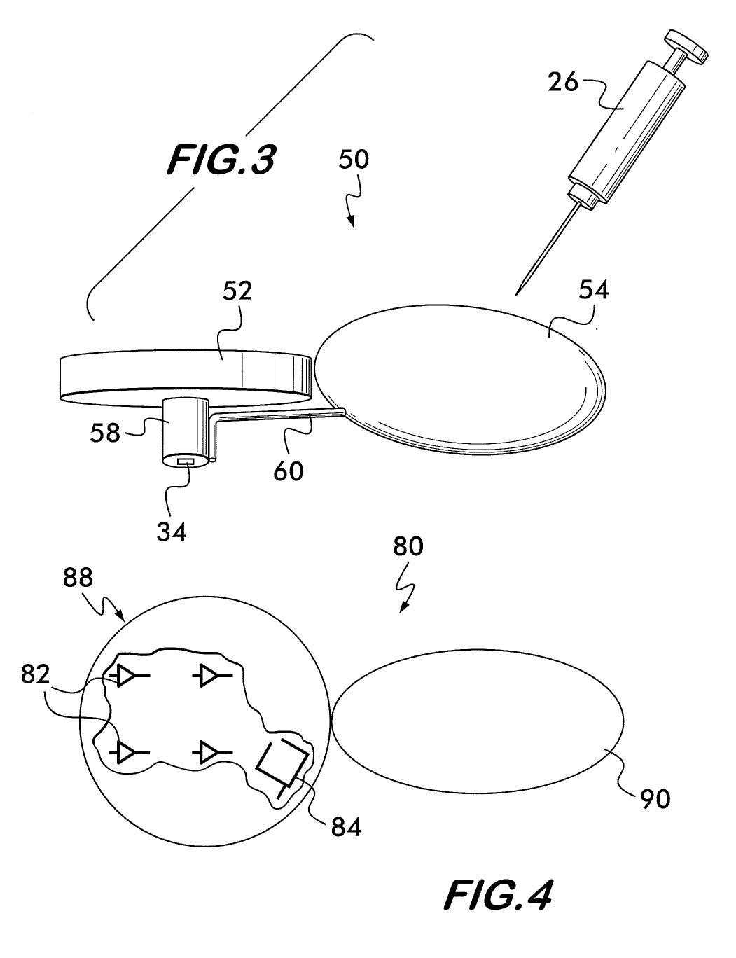

Referring now to FIG. 3, there is shown the intracranial pressure monitor system 50. The intracranial pressure monitor system 50 is an alternate embodiment of the intracranial monitoring system 10. The system 50 includes a housing portion 52 and an extending portion 58, which can be substantially similar to the housing portion 18 and the extended portion 32. The extending portion 58 can extend through an opening in the skull 14 and the dura 38, and position a sensor 34 directly in contact with the CSF within the subarachnoid space 44, as previously described.

A reservoir 54 provided in the intracranial pressure monitor system 50 can be located between the scalp 16 and the skull 14, as previously described with respect to the reservoir 22. Additionally, the reservoir 54 can be a self sealing elastic bladder, and the interior of the reservoir 54 can be in fluid communication with the CSF by way of a tube 60. The tube 60 therefore permits removing CSF from the inside of the skull 14, and injecting fluid into the CSF within the intracranial pressure monitor system 50. However, in order to provide the fluid communication between the reservoir 54 and the CSF, the tube 60 of the intracranial pressure monitor system 50 can pass through an opening in the housing portion 52 or through an opening in the extending portion 58. The tube 60, or an extension of the tube 60, therefore extends along the interior of at least a lower portion of the extending portion 58, to come in contact with the CSF, and bring the sensor 34 into direct contact with the CSF. Additionally, it is possible to palpate the reservoir 54 from outside the scalp 16. It is also possible to withdraw fluid from or inject fluid into the reservoir 54 using the syringe 26.

Referring now to FIG. 4, there is shown a top view of an intracranial pressure monitor system 80. The intracranial pressure monitor system 80 includes a housing portion 88 and a reservoir 90, as previously described. A tube for providing fluid communication between the reservoir 90 and the CSF can also be provided. Additionally, the intracranial pressure monitor system 80 includes an array of energy transducers 82. The energy transducers 82 can be any type of energy transducers for receiving light energy and converting the light energy into electrical energy, for example they can be photodetectors, photodiodes, photocells, solar cells, etc. The electrical energy provided by the transducers 82 can be used for operating the circuitry of the intracranial pressure monitor system 80. Alternately, the electrical energy provided by the transducers 82 can be used for recharging the battery or capacitor within the housing portion 88, for operating the circuitry of the intracranial pressure monitor system 80. The antenna 84 can be used to transmit a signal representing the pressure of the CSF to the exterior of the scalp for display.

The array of transducers 82 can be attached to the upper surface of the housing portion 88, or to a substrate, which can be attached to the upper surface of the housing portion 88. If the scalp is not closed over the housing portion 88, light can reach the transducers 82 directly, and be converted into electrical energy. If the scalp is closed over the housing portion 88, sufficient light can penetrate through the scalp to reach the transducers 82, and be converted into electrical energy. Alternately, the scalp can be closed and the substrate can be located outside the scalp. In this case, the electrical energy provided by the transducers 82 must be coupled to the housing portion 88 through the scalp, e.g. by wires passing through the scalp. In any case, the number of transducers 82 provided in the intracranial pressure monitor system 80 must be sufficient to provide the amount of electrical energy required under the lighting conditions which prevail during operation of the intracranial pressure monitor system 80.

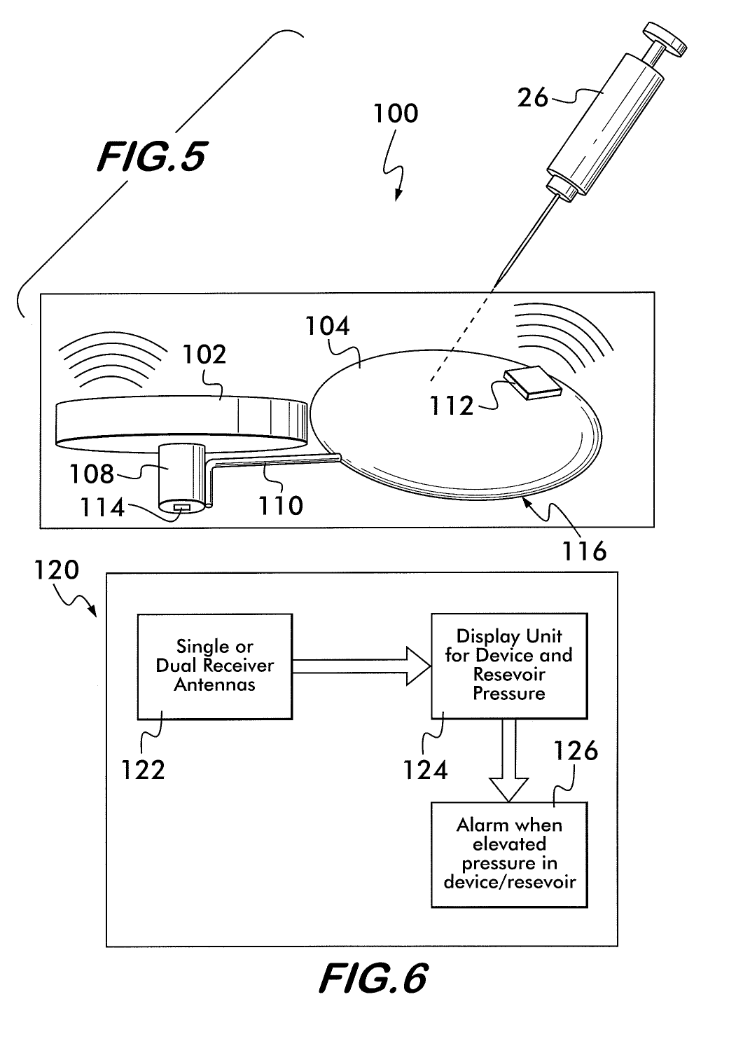

Referring now to FIGS. 5, 6, there are shown the intracranial pressure monitor system 100, and the receiving and feedback subsystems 120 associated with the intracranial pressure monitor system 100. The intracranial pressure monitor system 100 includes the housing portion 102 with an extending portion 108. The extending portion 102 depends downwardly from the housing portion 102 and passes through openings drilled through the skull and dura. The extending portion 108 includes a sensor 114 coupled to its tip which can be placed in direct contact with the CSF within the skull of a person being monitored. The sensor 114 thus provides a signal representative of a directly detected pressure measurement of the CSF. The pressure signal from the sensor 114 is conducted through the extending portion 108 to the housing 102 by wires or a coaxial cable.

A fluid reservoir 104 can be included in the intracranial pressure monitor 100. The interior of the fluid reservoir 104 can be in fluid communication with the CSF by way a tube 110 passing through the openings in the skull and dura. The tube 110 can extend directly through the openings in the skull and dura to the CSF. Alternately, the tube 110 can be coupled to an opening in the housing portion 102 or the extending portion 108, and come into fluid communication with the CSF by way of the bottom of the extending portion 108.

Additionally, the fluid reservoir 104 can be provided with a sensor 112. The sensor 112 can be substantially similar to the sensor 34, and can be within the fluid reservoir 104, or attached to an inner surface or an outer surface of a wall of the fluid reservoir 104. Alternately, the sensor 112 can be embedded within a wall of the fluid reservoir 104. Thus, the pressure or the changes in the pressure of the fluid within the fluid reservoir 104 can be detected by the sensor 112. The pressure or changes in the pressure according to the sensor 104 can modulate a carrier frequency in a manner similar to the manner used to modulate a carrier frequency with the signals provided by the sensors 34, 114. The signal from the sensor 104 can be used in addition to or in place of a pressure signal from the sensor 114.

If a sensor 112 is included in an embodiment having a sensor 114, signals from the two sensors 112, 114 can share a single frequency. Alternately, the signal from the sensor 114 can modulate a first frequency (for example 2.45 GHz) and the signal from the sensor 112 can modulate a second frequency (for example 5 GHz). In a preferred embodiment of the invention, a threshold modulating signal can be established in a receiver to generate an alarm for a healthcare provider, and initiate a fluid withdrawal from the fluid reservoir 104 using the syringe 26.

The receiving and feedback subsystems 120 can therefore include single or dual receiver antennas for receiving the signals from the sensor 112 and/or the sensor 114, as shown in block 122. A display unit can display one or both pressure readings, as shown in block 124 for monitoring by a healthcare provider. As previously described, an alarm can be provided when a threshold elevated pressure is detected at the extending portion 108 or in the fluid reservoir 104, as shown in block 126.

While the invention has been described in detail and with reference to specific examples thereof, it will be apparent to one skilled in the art that various changes and modifications can be made therein without departing from the spirit and scope thereof.

What is claimed is:

1. An intracranial pressure device for measuring CSF pressure in a skull having a scalp, comprising:
a housing located between the scalp and the skull containing pressure device circuitry;
a conduit extending downwardly from the housing to the vicinity of the CSF; and
a pressure sensor coupled to the conduit and located in communication with the CSF wherein the pressure sensor senses the pressure of the CSF and provides a signal representative of the pressure of the CSF to the pressure device circuitry located in the housing by way of the conduit.

2. The intracranial pressure device of claim 1, wherein the skull has a dura and the conduit extends by way of an opening through the skull and an opening through the dura to position the sensor in direct contact with the CSF.

3. The intracranial pressure device of claim 1, further comprising a fluid reservoir in communication with the CSF by way of a tube.

4. The intracranial pressure device of claim 1, further comprising a fluid reservoir in communication with the CSF by way of the housing.

5. The intracranial pressure device of claim 3, wherein the fluid reservoir contains CSF.

6. The intracranial pressure device of claim 3, wherein the fluid reservoir is self sealing after it is penetrated by a syringe to prevent fluid from passing through a wall of the fluid reservoir at a point where the fluid reservoir is penetrated by the syringe.

7. The intracranial pressure device of claim 5, wherein fluid is withdrawn from the fluid reservoir by way of a syringe.

8. The intracranial pressure device of claim 5, wherein fluid is injected into the fluid reservoir by way of a syringe.

9. The intracranial pressure device of claim 7, wherein the fluid is withdrawn from the fluid reservoir in accordance with the signal representative of the pressure of the CSF.

10. The intracranial pressure device of claim 1, wherein the sensor is MEMS-based circuitry.

11. The intracranial pressure device of claim 1, wherein the pressure device circuitry comprises transmission circuitry and a transmission signal is transmitted by the transmission circuitry in accordance with the signal representative of the pressure of the CSF.

12. The intracranial pressure device of claim 11, further comprising a display provided in accordance with the transmission signal.

13. The intracranial pressure device of claim 1, wherein the pressure device circuitry includes a battery further comprising an energy source for recharging the battery.

14. The intracranial pressure device of claim 13, wherein the energy source comprises an energy transducer.

15. The intracranial pressure device of claim 14, wherein the energy transducer comprises a photodetector for converting light energy into electrical energy.

16. The intracranial pressure device of claim 13, further comprising a substrate carrying a plurality of energy transducers.

17. The intracranial pressure device of claim 14, wherein the energy transducer is attached to the housing portion.

18. The intracranial pressure device of claim 17, wherein the energy transducer is located interior to at least a layer of the skin.

19. The intracranial pressure device of claim 3, further comprising a sensor applied to the fluid reservoir to provide a signal representative of the pressure of the fluid within the fluid reservoir.

20. The intracranial pressure device of claim 3, wherein the fluid reservoir is in fluid communication with intraventricular CSF by way of the tube.

21. The intracranial pressure device of claim 1, further comprising a shunt which is in fluid communication with an intraventricular space for draining the ventricular space.
